(19) United States
(12) Patent Application Publication
(10) Pub. No.: US 2024/0354938 A1
(43) Pub. Date: Oct. 24, 2024

(54) Systems and Methods for Prediction of Hematoma Expansion Using Automated Deep Learning Image Analysis

(71) Applicant: Northwestern University, Evanston, IL (US)

(72) Inventors: Andrew M. Naidech, Evanston, IL (US); Yuan Luo, Chicago, IL (US)

(21) Appl. No.: 18/569,070

(22) PCT Filed: Jun. 10, 2022
(86) PCT No.: PCT/US22/33015

§ 371 Date: Dec. 11, 2023

Related U.S. Application Data
(60) Provisional application No. 63/209,533, filed on Jun. 11, 2021.

(51) Int. Cl. G06T 7/00; G16H 50/20

(52) U.S. Cl. CPC G06T 7/0012; G16H 50/20; G06T 2207/10081; G06T 2207/20021; G06T 2207/20076; G06T 2207/20081; G06T 2207/30008; G06T 2207/30016

(57) ABSTRACT

A computing device for prediction of hematoma expansion using deep learning techniques includes a processor and memory in communication with the processor and storing instructions that, when read by the processor, cause the computing device to: retrieve, from a data store, electronic data corresponding to one or more diagnostic scans of a patient, preprocess the one or more diagnostic scans of the patient, perform deep learning analysis based on deep learning model trained to classify hematoma expansion, predict, based on the deep learning analysis of the one or more diagnostic scans, a probability of hematoma expansion for the patient, and provide, via a display, the probability of hematoma expansion for the patient as a combination of a heat map and a diagnostic scan of the one or more diagnostic scans of the patient.

### CROSS-REFERENCE TO RELATED APPLICATION(S)

This application claims the benefit of, and priority to, U.S. Provisional Patent Application No. 63/209,533 filed on Jun. 11, 2021, the content of which is expressly incorporated herein by reference in its entirety for any and all non-limiting purposes.

### STATEMENT OF GOVERNMENT INTERESTS

This invention was made with government support under NIH NS110779 awarded by National Institutes of Health. The government has certain rights in the invention.

### FIELD OF USE

Aspects of the disclosure relate generally to neuroimaging analysis and more specifically to predicting hematoma expansion via deep learning image analysis.

### BACKGROUND

Intracerebral hemorrhage (ICH) is the deadliest form of stroke. Hematoma expansion is a prominent preventable cause of poor outcomes after spontaneous ICH. The appearance of the hematoma on neuroimaging (e.g., hypodensities, irregular shape) may predict hematoma expansion after the diagnostic computed tomography (CT) scan. We developed and tested a deep learning algorithm to predict subsequent hematoma expansion.

### SUMMARY

The following presents a simplified summary of various aspects described herein. This summary is not an extensive overview, and is not intended to identify key or critical elements or to delineate the scope of the claims. The following summary merely presents some concepts in a simplified form as an introductory prelude to the more detailed description provided below. Corresponding apparatus, systems, and computer-readable media are also within the scope of the disclosure.

Systems and methods in accordance with this disclosure can use deep learning models to analyze diagnostic scans, such as CT scans, based on a deep learning model to identify a likelihood of hematoma expansion. The deep learning may improve the precision of identifying patients that would benefit from receiving interventions intended to reduce hematoma expansion to improve the speed and precision of interventions to improve patient outcomes.

The systems and methods described in this disclosure will also help with generating accurate predictions of hematoma expansion from neuroimaging that, in turn, could lengthen a time window for patient selection by using the deep learning prediction rather than time from symptom onset as an inclusion criterion.

These features, along with many others, are discussed in greater detail below.

### BRIEF DESCRIPTION OF THE DRAWINGS

The present disclosure is described by way of example and not limited in the accompanying figures in which like reference numerals indicate similar elements and in which:

FIG. 1 shows an example of a processing system according to one or more aspects of the disclosure;

FIG. 2 shows an example of a computing device according to one or more aspects of the disclosure;

FIG. 3A shows an illustrative process for predicting hematomas according to one or more aspects of the disclosure;

FIG. 3B shows an illustrative process for generating hematoma labels according to one or more aspects of the disclosure;

FIG. 4 shows an illustrative deep learning framework according to one or more aspects of the disclosure;

FIG. 5 shows an illustrative receiver operating characteristic curve according to one or more aspects of the disclosure; and

FIG. 6 shows illustrative CT Scan and heatmap images according to one or more aspects of the disclosure.

### DETAILED DESCRIPTION

In the following description of the various embodiments, reference is made to the accompanying drawings, which form a part hereof, and in which is shown by way of illustration various embodiments in which aspects of the disclosure can be practiced. It is to be understood that other embodiments can be utilized and structural and functional modifications can be made without departing from the scope of the present disclosure. Aspects of the disclosure are capable of other embodiments and of being practiced or being carried out in various ways. In addition, it is to be understood that the phraseology and terminology used herein are for the purpose of description and should not be regarded as limiting. Rather, the phrases and terms used herein are to be given their broadest interpretation and meaning.

By way of introduction, aspects discussed herein can relate to methods and techniques for predicting hematoma expansion using deep learning analysis. Intracerebral hemorrhage (ICH) is the deadliest form of stroke. Hematoma (or haematoma) expansion is a prominent preventable cause of poor outcomes after spontaneous ICH. The appearance of the hematoma on neuroimaging (e.g., hypodensities, irregular shape) predicts hematoma expansion after the diagnostic computed tomography (CT) scan. Aspects of the disclosure discuss use of and testing of a deep learning algorithm to predict subsequent hematoma expansion.

Intracerebral hemorrhage (ICH) is the deadliest form of stroke. A rupture of an artery leads to extravasation of blood into brain tissue. The larger the intracranial hematoma, the worse the patient's outcome is likely to be. Hematoma expansion, e.g., growth of the hematoma after a diagnostic computed tomography (CT) scan, may occur within hours of symptom onset. Small amounts of hematoma expansion may be disabling or deadly. However, predicting hematoma expansion remains inaccurate, hampering efforts to develop new interventions. Clinical trials to improve patient outcomes by reducing hematoma expansion have had mixed success. Potential harms to both hemostatic treatment (e.g., thrombosis) and blood pressure reduction (e.g., acute renal failure) exist, so identifying the patients most likely to benefit from such treatments is crucial to maximize potential benefits and to minimize potential risks. As such, a need has been recognized to provide faster and more accurate methods to select patients for interventions to reduce hematoma expansion.

In some cases hematoma expansion may be predicted from its appearance on the diagnostic (e.g., first) CT scan. Hypodensities in the hematoma, an irregular shape, and/or heterogeneous density all may signal a higher likelihood of subsequent hematoma expansion. However, such predictions may not be possible at a location and/or a time needed by patients with ICH present. Systems and methods in accordance with this disclosure utilize an automated algorithm that could identify patients likely to have hematoma expansion and may help target interventions to reduce hematoma expansion and/or improve patient outcomes.

Automatically identifying patients likely to have hematoma expansion from neuroimaging is theoretically feasible features may be recognizable by convolutional neural networks, a type of deep learning that abstracts shapes and borders from data. Testing of the hypothesis that deep learning could predict hematoma expansion from CT in patients with acute ICH is described below in greater detail.

### Operating Environment and Computing Devices

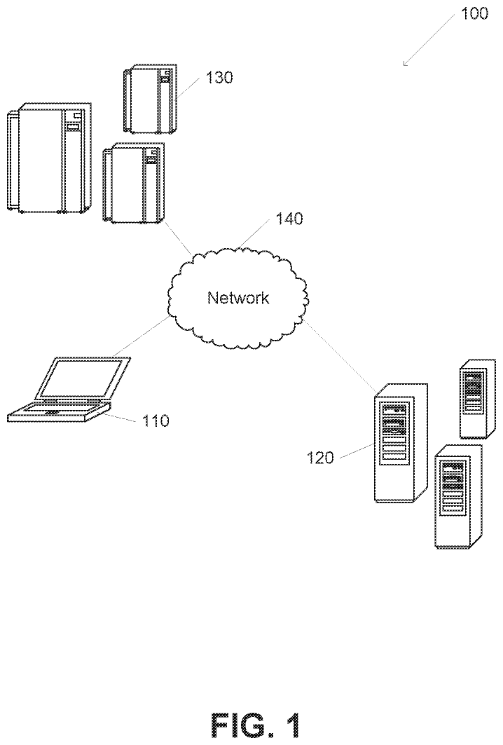

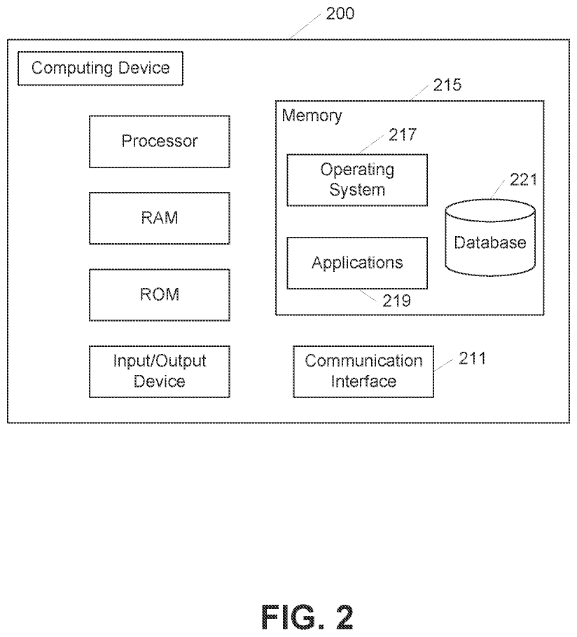

FIG. 1 shows an operating environment 100. The operating environment 100 can include at least one client device 110, at least one database system 120, and/or at least one server system 130 in communication via a network 140. The network 140 can include a local area network (LAN), a wide area network (WAN), a wireless telecommunications network, and/or any other communication network or combination thereof. It will be appreciated that the network connections shown are illustrative and any means of establishing a communications link between the computers can be used. The existence of any of various network protocols such as TCP/IP, Ethernet, FTP, HTTP and the like, and of various wireless communication technologies such as GSM, CDMA, WiFi, and LTE, is presumed, and the various computing devices described herein can be configured to communicate using any of these network protocols or technologies. Any of the devices and systems described herein can be implemented, in whole or in part, using one or more computing devices described with respect to FIG. 2.

Client devices 110 can obtain and/or process CT scans and/or other clinical data as described herein. Database systems 120 can obtain, store, and provide CT scans and/or other clinical data as described herein. Databases can include, but are not limited to relational databases, hierarchical databases, distributed databases, in-memory databases, flat file databases, XML databases, NoSQL databases, graph databases, and/or a combination thereof. Server systems 130 can obtain and/or process CT scans and/or other clinical data as described herein.

The data transferred to and from various computing devices in the operating environment 100 can include secure and sensitive data, such as confidential documents, personally identifiable information, protected health information (PHI), and account data. Therefore, it can be desirable to protect transmissions of such data using secure network protocols and encryption, and/or to protect the integrity of the data when stored on the various computing devices. For example, a file-based integration scheme or a service-based integration scheme can be utilized for transmitting data between the various computing devices. Data can be transmitted using various network communication protocols. Secure data transmission protocols and/or encryption can be used in file transfers to protect the integrity of the data, for example, File Transfer Protocol (FTP), Secure File Transfer Protocol (SFTP), and/or Pretty Good Privacy (PGP) encryption. In many embodiments, one or more web services can be implemented within the various computing devices. Web services can be accessed by authorized external devices and users to support input, extraction, and manipulation of data between the various computing devices in the operating environment 100. Web services built to support a personalized display system can be cross-domain and/or cross-platform, and can be built for enterprise use. Data can be transmitted using the Secure Sockets Layer (SSL) or Transport Layer Security (TLS) protocol to provide secure connections between the computing devices. Web services can be implemented using the WS-Security standard, providing for secure SOAP messages using XML encryption. Specialized hardware can be used to provide secure web services. For example, secure network appliances can include built-in features such as hardware-accelerated SSL and HTTPS, WS-Security, and/or firewalls. Such specialized hardware can be installed and configured in the operating environment 100 in front of one or more computing devices such that any external devices can communicate directly with the specialized hardware.

Turning now to FIG. 2, a computing device 200 that can be used with one or more of the computational systems is described. The computing device 200 can include a processor 203 for controlling overall operation of the computing device 200 and its associated components, including RAM 205, ROM 207, input/output device 209, communication interface 211, and/or memory 215. A data bus can interconnect processor(s) 203, RAM 205, ROM 207, memory 215, I/O device 209, and/or communication interface 211. In some embodiments, computing device 200 can represent, be incorporated in, and/or include various devices such as a desktop computer, a computer server, a mobile device, such as a laptop computer, a tablet computer, a smart phone, any other types of mobile computing devices, and the like, and/or any other type of data processing device.

Input/output (I/O) device 209 can include a microphone, keypad, touch screen, and/or stylus through which a user of the computing device 200 can provide input, and can also include one or more of a speaker for providing audio output and a video display device for providing textual, audiovisual, and/or graphical output. Software can be stored within memory 215 to provide instructions to processor 203 allowing computing device 200 to perform various actions. For example, memory 215 can store software used by the computing device 200, such as an operating system 217, application programs 219, and/or an associated internal database 221. The various hardware memory units in memory 215 can include volatile and nonvolatile, removable and non-removable media implemented in any method or technology for storage of information such as computer-readable instructions, data structures, program modules, or other data. Memory 215 can include one or more physical persistent memory devices and/or one or more non-persistent memory devices. Memory 215 can include, but is not limited to, random access memory (RAM) 205, read only memory (ROM) 207, electronically erasable programmable read only memory (EEPROM), flash memory or other memory technology, optical disk storage, magnetic cassettes, magnetic tape, magnetic disk storage or other magnetic storage devices, or any other medium that can be used to store the desired information and that can be accessed by processor 203.

Communication interface 211 can include one or more transceivers, digital signal processors, and/or additional circuitry and software for communicating via any network, wired or wireless, using any protocol as described herein.

Processor 203 can include a single central processing unit (CPU), which can be a single-core or multi-core processor, or can include multiple CPUs. Processor(s) 203 and associated components can allow the computing device 200 to execute a series of computer-readable instructions to perform some or all of the processes described herein. Although not shown in FIG. 2, various elements within memory 215 or other components in computing device 200, can include one or more caches, for example, CPU caches used by the processor 203, page caches used by the operating system 217, disk caches of a hard drive, and/or database caches used to cache content from database 221. For embodiments including a CPU cache, the CPU cache can be used by one or more processors 203 to reduce memory latency and access time. A processor 203 can retrieve data from or write data to the CPU cache rather than reading/writing to memory 215, which can improve the speed of these operations. In some examples, a database cache can be created in which certain data from a database 221 is cached in a separate smaller database in a memory separate from the database, such as in RAM 205 or on a separate computing device. For instance, in a multi-tiered application, a database cache on an application server can reduce data retrieval and data manipulation time by not needing to communicate over a network with a back-end database server. These types of caches and others can be included in various embodiments, and can provide potential advantages in certain implementations of devices, systems, and methods described herein, such as faster response times and less dependence on network conditions when transmitting and receiving data.

Although various components of computing device 200 are described separately, functionality of the various components can be combined and/or performed by a single component and/or multiple computing devices in communication without departing from the invention.

### Hematoma Expansion Prediction

Systems and methods in accordance with aspects of the disclosure predicts hematoma expansion after the diagnostic computed tomography (CT) scan using a deep learning algorithm to predict subsequent hematoma expansion.

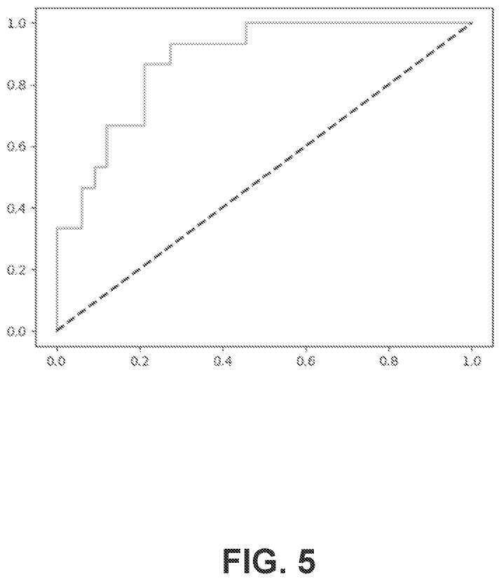

Demographic and neuroimaging data may be retrieved from patients enrolled in the various trials, such as recombinant factor VIIa phase II and the Factor VIIa for Acute Stroke Treatment (FAST, phase III) trials who received placebo. Additional patients who met criteria for the trials were prospectively identified at a single center. Hematoma volume was measured with computerized planimetry. Significant hematoma expansion was defined as 3 mL. The data was split into training (80%) and test (20%) data sets, where 20% of the training data was used for validation during model tuning. Deep learning algorithms were trained with standard software (Python V 3.6.8, PyTorch V 1.2.0). The performance was measured with the area under the receiver operating curve (AUROC), see FIG. 5.

Findings based on data discussed above are summarized as follows. From the two trials, data were available for 192 patients, of whom 62 (32%) had hematoma expansion. An additional 80 patients met the inclusion criteria from the single center, of whom 33 (41%) had hematoma expansion. The mean age was 63.5 years, the median Glasgow Coma Scale score was 14.5, the median NIH Stoke Scale score was 12.5, and the median baseline ICH volume was 11.4 ml. In the test set, the AUROC was 0.88 (95% Cl 0.73-0.92), with specificity 0.73, and sensitivity 0.93.

Interpretation of the findings shows Deep learning of non-contrast CT images can identify patients at risk for hematoma expansion after ICH. Automated CT image analysis is a feasible method to select patients for hemostatic therapy in acute ICH.

### Patients and Data Collection

Data was electronically retrieved from two randomized clinical trials, the recombinant factor VIIa phase II and the Factor VIIa for Acute Stroke Treatment (FAST, phase III), via the Virtual Online Stroke Trials Archive (VISTA). Provided data included hematoma volumes by standard, semi-automated techniques, and neuroimaging data that were collected during the clinical trials. Secondly, patients were identified at our site who met the inclusion criteria for the recombinant factor VIIa trials and were recruited into a prospective registry of patients (Northwestern University Brain Attack Registry, NUBAR) with ICH diagnosed with CT by a board-certified neurologist. All patients or a legally authorized representative (LAR) provided written informed consent for the use of electronic health data, with the exception of patients who were permanently comatose, died, or who could not consent and had no available LAR, in which case the Institutional Review Board granted a waiver from informed consent. Hematoma volumes were measured with a validated automated deep learning framework, validated against a previously measured standard voxel-based technique.

### Data Pre-Processinq

First, axial scans were selected with body part examined as “head.” Then the first scans (diagnostic scan) and a follow-up scan 1 to 96 hours after the diagnostic scan were identified for each patient. All DICOM files were then converted to NIfTI files using a publicly available resource dcm2niix v1.0.20201102. Hematoma volumes are readily available for FAST patients. While we employed an automatic deep learning pipeline to measure hematoma volumes for the NUBAR patients, we validated the automated measurement of hematoma volumes on 309 hematoma volumes previously measured using a semi-automated voxel-based technique. The Lin's Concordance Coefficient was 0.896 between the measured values and the ground truths. The automatic algorithm for hemorrhage volume measurement was then applied to obtain the ICH volumes for remaining diagnostic and follow-up scans not previously measured manually by semi-automated techniques for the NUBAR patients. The patients with hematoma volume growth on the follow-up scans greater or equal to 3 mL were labeled as positive for hematoma expansion, a previously validated standard. Data from the two clinical trials (the recombinant factor VIIa phase II and the Factor VIIa for Acute Stroke Treatment (FAST, phase III)) and the registry (NUBAR) were mixed to ensemble a diverse cohort of ICH patients.

All head CT scans were resized to 224 by 224 pixels in order to fit the input sizes of the pre-trained models. Each set of CT scans were then resliced to 0.5 mm slices, the standard for supra-tentorial slices at our institution. The scans with less than 18 slices after re-slicing (e.g., technically inadequate studies) were excluded from the analysis. For scans with more than 18 slices, we kept the top 18 slices that contains the most pixels of the hemorrhagic area. For each slice, three windows, brain window (I=40, w=80), bone window (I=500, w=3000) and subdural window (I=175, w=50) were created based on pixel data. By using different windows, various tissues were separated so that abnormal bleedings were easier for the algorithm to capture.

### Deep Learning Model Training

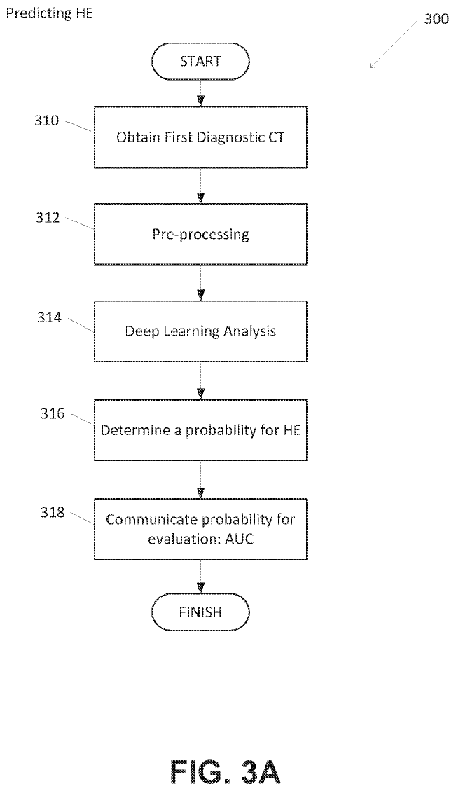

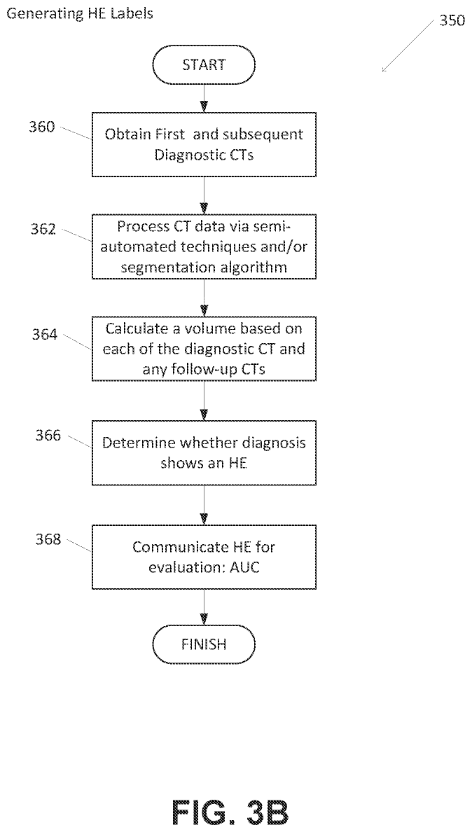

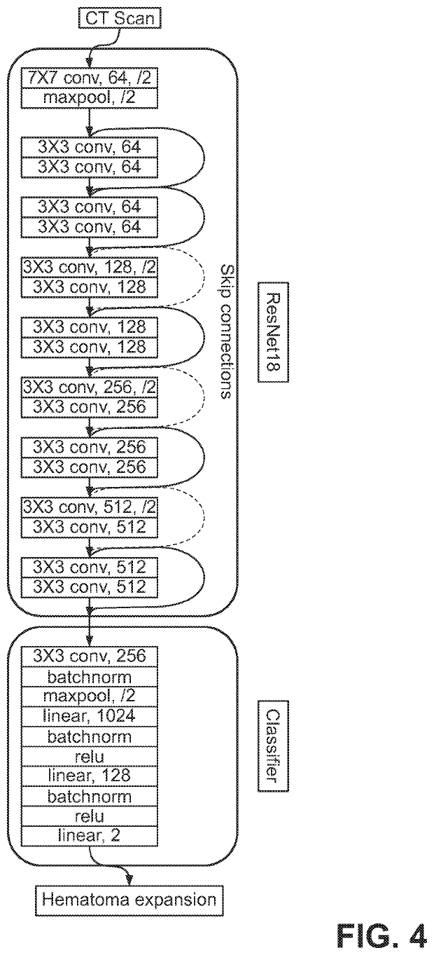

Supervised deep learning were performed for hematoma expansion after the diagnostic CT scan (input), with hematoma expansion as the label (classifier), no other clinical features were incorporated in building the model. The algorithm was intended to classify images as likely to have subsequent hematoma expansion ≥3 mL, a reliable predictor of poor patient outcomes. Before feeding into the deep learning architectures, the data were randomly split into training (64%), validation (16%), and test (20%) data sets stratified on labels. The training set was used to train the weights of the deep learning architecture, while the validation set was applied to select hyperparameters. The test set was held out until the final evaluation. Data was combined from both the trials and the single center data for generalization, and there were slight changes in practice over the years since the phase II recombinant factor VIIa trial and FAST were conducted. A ResNet1819 architecture was modified and fine-tuned with the weights initialized from the architecture pre-trained on ImageNet. To better incorporate the data, the final classifier layers were customized. Instead of one fully connected layer in the original architecture, a classifier constructed was applied by convolutional, pooling normalization, activation, and fully connected layers. The entire workflow is illustrated in FIGS. 3A and 3B, and a diagram of the modified deep learning network is shown in FIG. 4 shows an illustration of the modified deep learning framework, where the terms are described as conv: convolutional layer; batchnorm: batch normalization layer; maxpool: max-pooling layer; linear: fully connected layer; relu: relu activation; numbers before the “conv” are filter sizes, numbers after the “conv” are number of filters, numbers after the “linear” are the output sizes of the layer.

The output of the deep learning model are probabilities, and Youden's J statistics was then applied to obtain the optimal cutoff threshold for the binary classification. The training was performed parallelly on 4 Tesla K40c GPUs. The batch size was 8, the loss function minimized was cross-entropy loss, the initial learning rate was 0.0001, Adam optimizer was used. Hyperparameters are tuned based on the performance on the validation set. In total, each model was set to be trained for 60 epochs with early stopping if the performance does not improve for 10 consecutive epochs. The training time for each model was approximately 2 hours.

### Statistical Analysis

Data are described with mean±standard deviation (SD), or median and interquartile range [Q1-Q3] as appropriate. Means between groups were compared with a t-test or analysis of variance, and distributions were compared with a Mann-Whitney U or Kruskal-Wallis H test as appropriate.

Accuracy and receiver operating curve statistics (e.g., area under the curve [AUC]) were measured, as shown in FIG. 5. Automated hemorrhage volume measurement was conducted with MATLAB V 2020a Update 4 (9.8.0.1417392), Python V 3.6.8, and TensorFlow V 2.3.1. Deep learning models for hematoma expansion prediction and the calculation of evaluation metrics were performed with Python V 3.6.8, PyTorch V 1.2.0, and Scikit-learn V 0.23.2.

### Results

From the recombinant factor VIIa trials (phase II and FAST), data was available for 192 patients after data cleaning, of whom 62 (32%) had hematoma expansion. An additional 80 patients met the inclusion criteria from the single center, of whom 33 (41%) had hematoma expansion. The entire cohort contained a total of 272 patients with acute ICH, as shown in Table 1.

TABLE 1
Demographics of 304 patients

| | | n | % |
|---|---|---|---|
| Sex | Male | 169 | 62.1 |
| | Female | 103 | 37.9 |

| | | |
|---|---|---|
| Age | Mean ± SD | 63.5 ± 12.8 |

| | | | |
|---|---|---|---|
| Ethnicity | American Indian/ | 1 | 0.4 |
| | Native Alaskan | | |
| | Asian | 53 | 19.5 |
| | Black or African- | 42 | 15.4 |
| | American | | |
| | Native Pacific | 3 | 1.1 |
| | Islander | | |
| | White | 169 | 62.1 |
| | Other | 4 | 1.5 |

| | | |
|---|---|---|
| Baseline NIHSS | Median [Q1-Q3] | 12.5 [6.5-17.0] |
| Baseline ICH | Median [Q1-Q3] | 11.4 [4.5-23.7] |
| volume | | |
| GCS score at | Median [Q1-Q3] | 14.5 [13.0-15.0] |

| | | | |
|---|---|---|---|
| admission | | | |
| Hematoma | Yes | 95 | 34.9 |
| expansion | No | 177 | 65.1 |
| Sex | Male | 169 | 62.1 |
| | Female | 103 | 37.9 |

| | | |
|---|---|---|
| Age | Mean ± SD | 63.5 ± 12.8 |

| | | | |
|---|---|---|---|
| Ethnicity | American Indian/ | 1 | 0.4 |
| | Native Alaskan | | |
| | Asian | 53 | 19.5 |
| | Black or African | 42 | 15.4 |
| | American | | |
| | Native Pacific | 3 | 1.1 |
| | Islander | | |
| | White | 169 | 62.1 |
| | Other | 4 | 1.5 |

| | | |
|---|---|---|
| Baseline NIHSS | Median [Q1-Q3] | 12.5 [6.5-17.0] |
| Baseline ICH | Median [Q1-Q3] | 11.4 [4.5-23.7] |
| volume | | |
| GCS score at | Median [Q1-Q3] | 14.5 [13.0-15.0] |

| | | | |
|---|---|---|---|
| admission | | | |
| Hematoma | Yes | 95 | 34.9 |
| expansion | No | 177 | 65.1 |

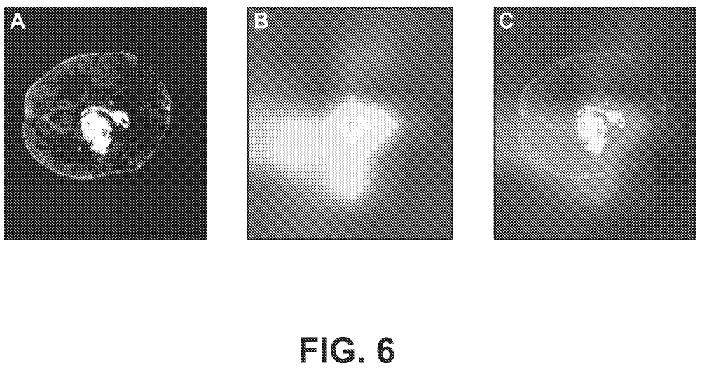

After fine-tuning, the deep learning algorithm yielded excellent performance (AUC=0.88, 95% Cl: 0.73-0.92) on predicting hematoma expansion from diagnostic head CT scans. The accuracy was 0.79, the specificity was 0.73, the sensitivity was 0.93, the F1 measure for the positive class (HE class) was 0.74, and the macro-averaged F1 measure was 0.78. The receiver operating characteristic curve is shown in FIG. 5. For a loaded set of processed CT scan, the trained model can give a prediction of hematoma expansion with in 0.5 s on our device. The resulting deep learning model was also capable of identifying the regions on the CT scans that contribute the most to the prediction. A widely-used localization algorithm, Grad-Cam was employed to highlight the hematoma as the region of interest for predicting subsequent hematoma expansion as shown in FIG. 6. In FIG. 6, panel A shows the original slice from a set of CT scan; panel B shows the heatmap generated by the deep learning algorithm during the prediction; and panel C shows the overlap of the CT scan and the heatmap.

### Discussion

The hypothesis that deep learning could identify patients likely to experience hematoma expansion in the first hours after spontaneous ICH was tested. Excellent accuracy was found in a cohort of several hundred patients, including two randomized, clinical trials and a prospectively identified registry. These results suggest that automated algorithms would be useful for quickly and precisely targeting interventions for patients with acute ICH in patients who are likely to have hematoma expansion after the diagnostic CT scan. In clinical use, algorithms like those we describe here could improve the speed and precision of interventions for ICH to reduce hematoma expansion, potentially improving patient outcomes.

Even among patients who present within a few hours of ICH symptom onset, predicting hematoma expansion remains challenging. As a result, imprecisely administered interventions will often be given to patients with no reasonable likelihood of benefit but who are still have a reasonable likelihood of adverse events (e.g., thrombosis). Deep learning could improve the precision of which patients receive interventions intended to reduce hematoma expansion. More precise patient selection for interventions would increase the likelihood of benefit while excluding patients unlikely to benefit.

Interventions for ICH are limited by time from symptom onset. Clinical research and trials for patients with ICH have progressively restricted the time window for subjects in an attempt to increase the precision of patient selection, typically between two and twelve hours from symptom onset. Accurate predictions of hematoma expansion from neuroimaging could lengthen the time window for patient selection by using a prediction of hematoma expansion from neuroimaging rather than time from symptom onset as an inclusion criterion. Deep learning to predict the risk of hematoma expansion could increase the number of patients who could potentially benefit.

The weights were initiated from the ResNet18 model pre-trained on ImageNet which in large is intended for the general domain, and generally performs well for medical applications. Pre-training may boost the performance of convolutional deep neural networks on medical images. One or more deep learning models created specifically for the purpose of predicting hematoma expansion may also be used.

In summary, deep learning was found to predict hematoma expansion with high accuracy in patients from the FAST trials and similar, prospectively identified patients. Future research might determine if similar approaches could precisely identify patients most likely to benefit from specific interventions to reduce hematoma expansion and improve outcomes after ICH.

### Abbreviations

ICH: intracerebral hemorrhage
CT: computed tomography
AUC: area under the curve in receiver operating characteristic analysis
LAR: legally authorized representative
SD: standard deviation
Q1: the first quartile
Q3: the third quartile
DICOM: Digital Imaging and Communications in Medicine
NIfTI: The Neuroimaging Informatics Technology Initiative
NSAIDs: nonsteroidal anti-inflammatory drugs
NOAC: novel oral anti-coagulants
LMWH: low-molecular-weight heparin
NIHSS: National Institutes of Health Stroke Scale
GCS: Glasgow Coma Scale

One or more aspects discussed herein can be embodied in computer-usable or readable data and/or computer-executable instructions, such as in one or more program modules, executed by one or more computers or other devices as described herein. Generally, program modules include routines, programs, objects, components, data structures, and the like, that perform particular tasks or implement particular abstract data types when executed by a processor in a computer or other device. The modules can be written in a source code programming language that is subsequently compiled for execution, or can be written in a scripting language such as (but not limited to) HTML or XML. The computer executable instructions can be stored on a computer readable medium such as a hard disk, optical disk, removable storage media, solid-state memory, RAM, and the like. As will be appreciated by one of skill in the art, the functionality of the program modules can be combined or distributed as desired in various embodiments. In addition, the functionality can be embodied in whole or in part in firmware or hardware equivalents such as integrated circuits, field programmable gate arrays (FPGA), and the like. Particular data structures can be used to more effectively implement one or more aspects discussed herein, and such data structures are contemplated within the scope of computer executable instructions and computer-usable data described herein. Various aspects discussed herein can be embodied as a method, a computing device, a system, and/or a computer program product.

Although the present invention has been described in certain specific aspects, many additional modifications and variations would be apparent to those skilled in the art. In particular, any of the various processes described above can be performed in alternative sequences and/or in parallel (on different computing devices) in order to achieve similar results in a manner that is more appropriate to the requirements of a specific application. It is therefore to be understood that the present invention can be practiced otherwise than specifically described without departing from the scope and spirit of the present invention. Thus, embodiments of the present invention should be considered in all respects as illustrative and not restrictive.

What is claimed is:

1. A computer-implemented method for prediction of hematoma expansion using machine learning techniques, comprising:
retrieving, from a data store, electronic data corresponding to one or more diagnostic scans of a patient;
preprocessing the one or more diagnostic scans of the patient;
performing, by a machine learning computing system, machine learning analysis based on machine learning model trained to classify hematoma expansion; and
predicting, based on the machine learning analysis of the one or more diagnostic scans, a probability of hematoma expansion for the patient.

2. The computer-implemented method of claim 1, wherein preprocessing the one or more diagnostic scans comprises one or both of of resizing the one or more diagnostic scans and re-slicing the one or more diagnostic scans.

3. The computer-implemented method of claim 1, further comprising providing, via a display, the probability of hematoma expansion for the patient as a combination of a heat map and a diagnostic scan of the one or more diagnostic scans of the patient.

4. The computer-implemented method of claim 3, wherein preprocessing the one or more diagnostic scans comprises discarding a diagnostic scan with less than 18 slices after re-sizing.

5. The computer-implemented method of claim 3, further comprising creating, based on pixel data and for each slice, a bone window, a brain window, and a subdural window.

6. The computer-implemented method of claim 1, wherein the predicting of the probability of hematoma expansion for the patient comprises classifying images as likely to have subsequent hematoma expansion greater than or equal to 3 mL.

7. The computer-implemented method of claim 1, wherein the machine learning computing system provides a prediction of hematoma expansion in less than 0.5 seconds.

8. A computing device for prediction of hematoma expansion using machine learning techniques, comprising:
a processor; and
a memory in communication with the processor and storing instructions that, when read by the processor, cause the computing device to:
retrieve, from a data store, electronic data corresponding to one or more diagnostic scans of a patient;
preprocess the one or more diagnostic scans of the patient;
perform machine learning analysis based on machine learning model trained to classify hematoma expansion; and
predict, based on the machine learning analysis of the one or more diagnostic scans, a probability of hematoma expansion for the patient.

9. The computing device of claim 8, wherein the instructions, when executed by the processor, cause the computing device to resize the one or more diagnostic scans, re-slice the one or more diagnostic scans, or resize and re-slice the one or more diagnostic scans.

10. The computing device of claim 8, wherein the instructions, when executed by the processor, cause the computing device to provide, via a display, the probability of hematoma expansion for the patient as a combination of a heat map and a diagnostic scan of the one or more diagnostic scans of the patient.

11. The computing device of claim 10, wherein the instructions, when executed by the processor, cause the computing device to discard a diagnostic scan with less than 18 slices after re-sizing.

12. The computing device of claim 10, wherein the instructions, when executed by the processor, cause the computing device to create, based on pixel data and for each slice, a bone window, a brain window, and a subdural window.

13. The computing device of claim 8, wherein the instructions, when executed by the processor, cause the computing device to classify mages as likely to have subsequent hematoma expansion greater than or equal to 3 mL.

14. The computing device of claim 8, wherein a prediction of hematoma expansion is provided in less than 0.5 seconds.

15. A non-transitory machine-readable medium storing instructions that, when executed by one or more processors, cause the one or more processors to perform steps comprising:
retrieving, from a data store, electronic data corresponding to one or more diagnostic scans of a patient;
preprocessing the one or more diagnostic scans of the patient;
performing, by a machine learning computing system, machine learning analysis based on machine learning model trained to classify hematoma expansion; and
predicting, based on the machine learning analysis of the one or more diagnostic scans, a probability of hematoma expansion for the patient.

16. The non-transitory machine-readable medium of claim 15, wherein preprocessing the one or more diagnostic scans comprises one or both of resizing the one or more diagnostic scans and re-slicing the one or more diagnostic scans.

17. The non-transitory machine-readable medium of claim 15, wherein the instructions further cause the one or more processors to perform a step of providing, via a display, the probability of hematoma expansion for the patient as a combination of a heat map and a diagnostic scan of the one or more diagnostic scans of the patient.

18. The non-transitory machine-readable medium of claim 17, wherein preprocessing the one or more diagnostic scans comprises discarding a diagnostic scan with less than 18 slices after re-sizing.

19. The non-transitory machine-readable medium of claim 17, cause the one or more processors to perform a step of creating, based on pixel data and for each slice, a bone window, a brain window, and a subdural window.

20. The non-transitory machine-readable medium of claim 15, wherein the predicting of the probability of hematoma expansion for the patient comprises classifying images as likely to have subsequent hematoma expansion greater than or equal to 3 mL.
